(19) United States
(12) Patent Application Publication
(10) Pub. No.: US 2009/0017093 A1
(43) Pub. Date: Jan. 15, 2009

(54) GELATINOUS MATERIAL FOR FILLING BONE AND/OR CARTILAGE DEFECTS

(76) Inventors: Marco Springer, Esslingen (DE); Arne Briest, Karlsruhe (DE)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/722,528

(22) PCT Filed: Dec. 22, 2005
(86) PCT No.: PCT/EP05/13925

§ 371 Date: Aug. 7, 2008

(30) Foreign Application Priority Data
Dec. 23, 2004 (DE) 102004063210.3
Apr. 13, 2005 (DE) 102005018283.6
Jul. 13, 2005 (DE) 102005034420.8

(51) Int. Cl. A61K 9/00

(52) U.S. Cl. 424/426

(57) ABSTRACT

Disclosed is a goal gel-type material for filling bone and/or cartilage defects. Said gel-type material comprises at least one gelatinizing component and collagen in at least one liquid dispersing agent. The collagen contains an active substance complex. Preferably, the inventive material is provided with osteoconductive and osteoinductive properties.

The invention relates primarily to a gelatinous material for filling bone and/or cartilage defects.

The treatment of bone defects is of considerable importance in medical care, especially in orthopedics, accident surgery, oral and dental surgery, and hand surgery. Although the natural bone itself has regenerative capabilities, which bring about induction of de novo bone and/or cartilage formation, ‘osteogenesis’, natural limits can be placed on this self-healing process by the size of a defect and/or functional disorders, for example metabolic disorders. The causes of the development of bone defects can be very different, for example they can be caused by cysts, atrophy, tumors etc. The focal point of treatment therefore increasingly lies in the reconstruction of the bone defect by suitable filling materials.

For filling bone defects, filling materials which are either poorly bioresorbable or not bioresorbable at all, or bioresorbable filling materials are usually used which compulsorily have a defined spatial shape. These bone replacement materials can be, for example, porous calcium phosphate ceramics, synthetic polymers, preferably polylactides or -glycolides, or ‘demineral-ized fillers’. The latter are mainly natural, in particular animal, preferably equine, porcine or bovine, bone tissue, in which the inorganic or mineral portion has been almost completely removed except for traces. An example of such a biomatrix is the lyophilized collagen extract of bovine bone of the Applicant, commercially obtainable under the name COLLOSS®. A further example of such a biomatrix is the lyophilized collagen extract of equine bone of the Applicant, commercially obtainable under the name COLLOSS®E. Owing to their structural biocompatibility, COLLOSS® and COLLOSS®E assist the bone growth and de novo bone formation process.

In the case of bone defects having poorly accessible cavities, in particular in the case of large defects, however, application of the filling materials mentioned is tedious and problematical with respect to the healing prospects. This also applies to a particular extent for comminuted fractures, in which the cavities to be filled are often very varied.

In a further development, the bone replacement material according to the prior art can be provided with a bone-forming protein. Thus, in EP 0 837 701, a compulsorily sterilized, implantable, bone-forming device is described which comprises an insoluble carrier material and a bone-forming protein. Although it is indicated in this specification that the device is applicable as a paste or gel by addition of a suitable binding material, it is to be inferred from the printed specification as a whole, in particular the examples, that it is a device having a defined spatial shape. The bone-forming protein should be able there to induce the development cascade of de novo bone formation. It is problematical, however, that the bone-forming properties are based exclusively on one individual protein. A possible loss of activity of this protein can therefore lead to a loss of the bone-forming properties of the material concerned. Moreover, such modified implants are often an only inadequate image of the conditions of de novo bone formation predominant in vivo, which is actually based on a complex interplay of many active substances.

The invention therefore sets itself the object of providing a novel, preferably injectable, osteo-inductive or bone- and/or cartilage-forming material, which essentially corresponds to the natural bone composition, is easily handleable and applicable and is also usable in a large volume to be filled.

This object is achieved by a material for filling bone and/or cartilage defects, as is described in claim 1. Preferred embodiments of this material are shown in claims 2 to 19. Molded articles which consist at least partially of the material according to the invention are listed in claims 20 and 21. Claims 22 to 27 relate to a suitable process for the production of a material or molded article of this type. Claims 28 to 33 relate to a kit for the treatment of bone and/or cartilage defects, which contains the material according to the invention. By way of reference, the wording of all claims is made part of the description.

The material according to the invention for filling bone and/or cartilage defects is a gelatinous material which comprises at least one gel-forming component and collagen in at least one liquid dispersant, the collagen containing an active substance complex. A gelatinous material should be understood as meaning any essentially shape-retaining, easily deformable disperse and liquid-containing system which contains at least one solid, colloidally disperse substance, a ‘gel-forming agent’ or a thickener, and at least one liquid, a ‘dispersant’. An active substance complex should be understood as meaning a combination of various active substances.

Preferably, the collagen is collagen of type 1. Preferentially, the collagen is coherent, i.e. it forms a spatial network in the dispersant, the individual collagen fibrils adhering to one another by means of covalent or noncovalent bonding forces at various points, ‘adhesive points’.

In a preferred embodiment, the dispersant is water or an aqueous medium and the material according to the invention is a ‘hydrogel-like material’. Preferably, the dispersant is an aqueous solution of at least one salt, preferably a physiological saline solution. According to another preferred embodiment, the dispersant is blood, in particular patient's blood, for example fresh blood, anticoagulated blood or a PRP (platelet rich plasma) fraction of blood. The constituents and factors present in the blood or in a blood fraction, in particular blood platelets and their released substances, can themselves have osteogenic, i.e. bone- and/or cartilage-forming, properties and in particular bring about an induction and/or increase in osteogenesis by interaction with the collagenic active substance complex.

According to the invention, it is in particular provided for the gel-forming component to be a hydrophilic polymer. A hydrophilic polymer should be understood as meaning a polymer which is able, due to the presence of at least one polar, preferably charged, atomic group, for example a carboxylate, sulfate and/or sulfonate function, to penetrate into water and to remain therein. In the broader sense, an ‘amphiphilic polymer’, i.e. a polymer which has both a hydrophilic and hydrophobic atomic group, should also be subsumed under the term hydrophilic polymer. In a preferred embodiment of the material according to the invention, the polymer is a polysaccharide or polysaccharide derivative. Preferably, it is a glycosaminoglycan (GAG), preferably hyaluronic acid or heparin, or a cellulose derivative, preferably carboxymethylcellulose (CMC), hydroxyethylcellulose (HEC), hydroxypropyl-cellulose (HPC) or ethylhydroxyethylcellulose (EHEC).

In another embodiment, depending on the dispersant used, alternative gel-forming agents, for example fats, paraffins or waxes, can also be used. This can be particularly advantageous if the dispersant is a not very polar liquid, preferably an appropriate organic solvent.

According to the invention, it is provided in particular for the concentration of the gel-forming component, in particular of the polysaccharide or polysaccharide derivative, preferably of hyaluronic acid or carboxymethylcellulose (CMC), to be between 0.5 and 25% by weight, in particular between 1 and 15% by weight, preferably between 1 and 10% by weight, based on the total volume of the gelatinous material.

In a particularly preferred embodiment, the collagen is present in its native conformation having triple-helical collagen fibrils. This is particularly advantageous, since the network of collagen fibers and/or fibrils is an ideal carrier material having osteoconductive or load-bearing and/or volume-stable properties. At the same time, the native collagen network offers suitable interstitial spaces for infiltration by bone cells or their precursor cells without first having to be brought artificially into a suitable spatial arrangement.

In another embodiment of the invention, the collagen can be present in denatured form, in particular acidically or basically treated, form. This can be advantageous in order to increase the solubility of the collagen for its further processing. In a refinement of the material according to the invention, the collagen can be a collagen which is originally denatured and subsequently renatured again.

According to the invention, it is provided for the osteoinductive character of the gelatinous material to be produced or increased by modification of the collagen using an active substance complex activating and/or stimulating osteogenesis. Preferably, the active substance complex is a complex of natural origin, i.e. the active substances contained in it are preferably present in their native form. The active substance complex of collagen is advantageously distinguished in that it contains ‘recruitment factors’, in particular chemotactics (chemotaxins), for example leukotrienes, which selectively induce the infiltration of the material according to the invention with body cells, preferably mesenchymal stem cells, cartilage precursor cells, fibroblasts and/or platelets.

Furthermore, it is preferred that the active substance complex contains ‘adhesion factors’, for example cytotactin, thenascin, laminin and/or fibronectin, for fixing the recruited or immigrated cells.

In a particularly preferred embodiment of the invention, the active substance complex of the gelatinous material moreover contains growth and/or maturation factors for the proliferation and differentiation of the immigrated cells, in particular cytokines. Preferably, the growth factors are bone growth factors, for example BMP (bovine morphogenetic protein), in particular BMP-2, BMP-7 and/or BMP-4, and IGF (insulin-like growth factor), in particular IGF 1, and TGF (transforming growth factor), in particular TGF β1. Preferably, the collagen alternatively or additionally contains FGF (fibroblast growth factor) and PDGF (platelet derived growth factor) as growth factors.

Owing to the provision of the collagen with this active substance complex, the collagen becomes a carrier for the osteoinductive information which optimally essentially makes available all active substances involved with the in vivo for de novo bone and/or cartilage formation. These active substances advantageously show overlapping activity functions, such that a possible loss of activity of one or more active substances can be taken over or compensated by the other active substances contained in the complex. Moreover, the application of a native active substance complex can be advantageous for the production of superadditive or synergistic effects in the reconstruction of a bone and/or cartilage defect by endogenous material. With respect to such natural active substance complexes, reference is made to EP 0 500 556.

In a particularly preferred manner, the “information carrier” with respect to the osteoinductivity is the already mentioned collagenic material COLLOSS® or COLLOSS®E, preferably COLLOSS®E, of the Applicant which, due to the preparation process, advantageously already contains an active substance complex of the described native active substances. Thus collagen and active substance complex form a native functional unit, which essentially agrees in its qualitative composition with the environment present in vivo and capable of de novo bone and/or cartilage formation.

Furthermore, it can be preferable that the active substance complex of the material according to the invention comprises a cytostatic. This is particularly advantageous in the case of cancerous changes in the area of the bone defect and/or of the surrounding tissue. In a further embodiment of the invention, the active substance complex comprises an antimicrobiotic, in particular antibiotic, active substance, in order to avoid or to suppress possible immune defense reactions in the body.

In a further embodiment of the invention, the different active substances of the complex mentioned by way of example can also be combined with other active substances, in particular synthetically, preferably recombinantly, prepared active substances, in order to achieve particularly advantageous effects, in particular superadditive or synergistic effects.

In addition to the embodiments already described, the material according to the invention can additionally contain at least one preferably structure-imparting filler. Advantageously, the filler, due to its spatial arrangement, offers additional surface areas for cell proliferation and/or differentiation. According to the invention, it can be provided for the filler to be present as granules. This is particularly preferred on account of the large specific surface area of granules, as in this way an adequate surface area for ingrowth of body cells, in particular bone and/or cartilage cells or their precursor cells, is made available. In a further embodiment of the invention, the filler is bioresorbable. In this way, a long-lasting stay of foreign materials in the body is avoided.

In a particularly preferred embodiment of the material according to the invention, the filler is porous. Preferably, the filler has an interconnecting porosity, i.e. the pore structure is optimized both between the individual particles of the filler and within the particles. The porous structuring of the filler is particularly advantageous for infiltration by body cells, in particular by bone and/or cartilage cells or their precursor cells. In a refinement of the invention, the filler is provided with the active substances described here and/or further, preferably pharmaceutical, active substances on the inner and/or outer surface of the particles. In this way, the active substances are optimally presented to the body, in particular the endogenous cells.

The pores of the filler preferably have pore diameters of at least 100 μm, in particular pore diameters between 100 and 300 μm, preferably of about 200 μm. This is particularly advantageous for the ingrowth of body cells, in particular bone and/or cartilage cells or their precursor cells, into the filler. In this way, the integration of the material according to the invention and its degradation or resorption is promoted. The filler can advantageously be tricalcium phosphate, hydroxyapatite or calcium sulfate. These filler materials are particularly preferred, since their ionic components form the main constituents of the natural mineral bone substance and are therefore a suitable biomimetic material.

In a particularly preferred embodiment, the filler is the material OSSAPLAST®, a bioresorbable and granulated β-tricalcium phosphate having high interconnecting porosity and a large specific surface area, commercially marketed by the Applicant.

It is furthermore possible for light metals to be used as fillers. Preferably, the light metals are magnesium, titanium or tantalum, which are particularly suitable on account of their resorbability, biocompatibility and easy handleability.

In a further embodiment, the material according to the invention is preferably chemically crosslinked. As a chemical crosslinker, a carbodiimide is particularly preferred, in particular N-(dimethylamino-propyl)-N′-ethylcarbodiimide (EDC), or glutaraldehyde.

According to the invention, it can moreover be provided for the material to be present sterilized or in sterilized form. The customary processes are suitable for sterilization. The sterilization of the material according to the invention by radioactive irradiation, preferably by gamma irradiation and/or ethylene oxide fumigation, is preferred.

In another embodiment of the invention, the material according to the invention is aseptically treated and/or prepared, in particular by use of sterile-filtered antibiotic solutions, preferably a sterile-filtered gentamicin solution. This can be particularly preferred, since in this way a possible disadvantageous influencing of the active substances contained and/or of the osteoinductive properties of the material according to the invention is avoided. Furthermore, in the case of a relatively gentle sterilization, in particular one not adversely affecting the osteo-inductivity of the material according to the invention, an additional aseptic treatment can be preferred.

According to the invention, the material is present in packaged form, in particular in sterile-packaged form. In a packaging of this type, the material can be stored for a relatively long period, in particular for a period of several months, without an adverse effect on the mode of action of the material according to the invention, in particular an adverse effect on its osteoinductive properties, occurring here. Suitable packaging materials are, in particular, plastic materials. The packaging materials can be designed as syringes, in particular as single-chamber or dual-chamber syringes.

The invention moreover comprises a molded article for filling bone and/or cartilage defects, which consists at least partially, preferably completely, of the material according to the invention, the dispersant being removed at least partially, preferably completely. Molded articles are to be understood as meaning aggregates of the gel-forming component and of the collagen containing an active substance complex, which optionally contain further components, for example binders, salts and/or preferably chemical crosslinkers. Preferably, these molded articles are present as three-dimensional molded articles, in particular as bars, rings or cylinders. Furthermore, it is possible for the molded articles to be present as cuboids, disks or the like. Molded articles of any desired shapes can be produced. Preferably, the molded article is freeze-dried for the removal of the dispersant. The molded articles thus obtained are advantageously convertible into a gelatinous material by addition of liquids, for example water or salt solutions, in particular a physiological saline solution. According to the invention, it is particularly preferred for the molded articles according to the invention to be convertible into a gelatinous material by addition of blood, in particular patient's blood. This reciprocal molded article—gelatinous material conversion advantageously makes possible a matching of the dimensional stability of the material or molded article according to the invention to the bone and/or cartilage defect to be treated in each case.

In another embodiment of the invention, a molded article having a putty-like consistency or composition is obtained by mere mixing of the gel-forming component, which is preferably present in powder form, and of the collagen (containing active substance complex), which is likewise preferably present in powder form. By addition of a dispersant, the gelatinous material according to the invention is formed.

The invention furthermore comprises a process for the production of the material or molded article according to one of the preceding claims, comprising the steps:
preparation of a mixture of a collagen containing an active substance complex, a gel-forming component and at least one liquid dispersant,
optionally addition of a preferably structure-imparting filler,
   for the provision of the gelatinous material and optionally for the provision of the molded article,
removal of the dispersant from the gelatinous material, preferably in a shape-imparting environment.

According to the invention, it can be provided for the collagen containing an active substance complex and/or the gel-forming component to be present in a dispersant during the preparation of the mixture. Preferably, the mixture is prepared from a suspension of the collagen in the dispersant and/or a solution of the gel-forming component in the dispersant. In a particularly preferred embodiment, an aqueous collagen suspension and/or an aqueous solution of the gel-forming component are mixed with one another.

In a further preferred embodiment, the collagen suspension is prepared from freeze-dried collagen (containing active substance complex). Advantageously, the collagen is a lyophilizate of the material COLLOSS® or COLLOSS®E, preferably COLLOSS®E, of the Applicant. In another embodiment of the process according to the invention, the collagen suspension is prepared from extracted collagen. The collagen extract is preferably an intermediate in the preparation of the material COLLOSS® or COLLOSS®E, preferably COLLOSS®E, of the Applicant.

According to the invention, it can in particular be provided for the collagen (containing active substance complex) and/or the gel-forming component for the preparation of the gelatinous material to be present or prepared in solid form, preferably as a lyophilizate and/or powder. According to the invention, it is particularly preferred if only the gel-forming component is present or prepared in solid form, preferably as a powder. Advantageously, the gel-forming component, which is preferably sterile, is initially introduced into a suitable vessel (vial) and blended, in particular stirred, with a collagen suspension, in particular a collagen suspension according to one and/or the embodiments of the process according to the invention previously described, which is preferably concentrated and/or sterile. The stirring process can take place manually or in automated form. The vessel can preferably be an open vessel, in particular a dish. According to the invention, it can be provided for the gel-forming component present in this form to be made available to the user, for example a doctor, in a vessel of this type, in particular a vessel which is open or to be opened. Depending on the desired viscosity of the gelatinous material to be prepared, provision can take place in different amounts. In a particularly preferred embodiment, a sterile COLLOSS® suspension or COLLOSS®E suspension, preferably COLLOSS®E suspension, is added to a sterile powder of a polysaccharide or polysaccharide derivative, in particular of a glycosaminoglycan (GAG), for example of hyaluronic acid or heparin, or of a cellulose derivative, for example of carboxymethyl-cellulose (CMC) initially introduced into an open vessel, in particular into a dish, and manually stirred, in particular until the formation of a homogeneous gelatinous material.

In a further preferred embodiment, the gelatinous material in particular prepared according to one of the preceding embodiments or the corresponding components for its preparation are transferred to and/or poured into an appropriate device, in particular a syringe, for example a single-chamber or dual-chamber syringe, or a vessel. This device is then made available to the user, for example a doctor. Preferably, a sterile and in particular concentrated COLLOSS® suspension or COLLOSS®E suspension, preferably COLLOSS®E suspension, and a sterile powder of a polysaccharide or polysaccharide derivative, in particular of a glycosaminoglycan (GAG), for example of hyaluronic acid or heparin, or of a cellulose derivative, for example of carboxymethylcellulose (CMC), are used in designs of this type.

According to the invention, it can also be preferred for the converted gelatinous material, which is in particular prepared according to one of the preceding embodiments, to be lyophilized. The lyophilizate resulting in this way can be dissolved again using a liquid, for example a physiological saline solution or blood, in particular patient's blood, and in particular converted to a gelatinous material. Advantageously, the lyophilizate is compressed before the addition of the liquid in order to obtain, after addition of liquid, a more highly concentrated gelatinous material with respect to the concentration of the gel-forming component and of the collagen containing an active substance complex. Preferably, the lyophilizate and/or more highly concentrated gel prepared in this way is prepared from a sterile and in particular concentrated COLLOSS® suspension or COLLOSS®E suspension, preferably COLLOSS®E suspension, and a sterile powder of a polysaccharide or polysaccharide derivative, in particular of a glycosaminoglycan (GAG), for example of hyaluronic acid or heparin, or of a cellulose derivative, for example of carboxymethylcellulose (CMC).

According to another embodiment, it is in particular preferred if both the collagen containing an active substance complex and the gel-forming component are present or prepared in solid form, in particular sterile form, the collagen preferably being present or prepared as a lyophilizate and the gel-forming component preferably being present or prepared as a powder. For the preparation of the material according to the invention, the collagen (containing active substance complex) and the gel-forming component can be initially introduced into a suitable vessel and, by the addition of the dispersant, preferably patient's blood, agitated or stirred to give a gelatinous mixture. The vessel for the preparation of the material according to the invention can, in particular, be closed, preferably screwed shut. The stirring of the mixture after addition of the dispersant can be carried out manually in a simple manner, in particular by a doctor, for example a surgeon. According to the invention, it can be provided for the agitated mixture to be incubated for a certain time, and subsequently to be agitated again. This can be particularly advantageous in order to improve the solubility and homogeneity of the collagen and of the gel-forming component, and of further components optionally present in the mixture. Alternatively to this, the mixture obtained after addition of the dispersant can also be immediately incubated and subsequently agitated.

Preferably, in the embodiment of the process according to the invention just described, the active substance complex-containing collagen used is the material COLLOSS® or COLLOSS®E, preferably COLLOSS®E, of the Applicant and the gel-forming component used is a polysaccharide or polysaccharide derivative, in particular a glycosaminoglycan (GAG), for example hyaluronic acid or heparin, or a cellulose derivative, for example carboxymethylcellulose (CMC).

In the case of the use of COLLOSS® or COLLOSS®E, preferably COLLOSS®E, for the preparation of the gelatinous material, in particular in a closable vessel, it can be advantageous to provide additional collagen in solid and in particular sterilized form, preferably as a powder. In this way, the osteo-conductive properties, i.e. the load-bearing and/or volume-stabilizing properties of the gelatinous material, can be additionally increased. Preferably, the collagen in solid form is collagen of type 1.

In a particularly preferred embodiment, COLLOSS® lyophilizate, powder of collagen type 1 and powder of a polysaccharide or polysaccharide derivative, in particular of a glycosaminoglycan (GAG), for example of hyaluronic acid or heparin, or of a cellulose derivative, for example of carboxymethylcellulose (CMC), are initially introduced into a vessel which can preferably be screwed shut and stirred to give the material according to the invention according to the embodiments described above after addition of the dispersant, in particular of patient's blood, optionally while carrying out an incubation.

In a further, particularly preferred embodiment, COLLOSS®E lyophilizate, powder of collagen type 1 and powder of a polysaccharide or polysaccharide derivative, in particular of a glycosaminoglycan (GAG), for example of hyaluronic acid or heparin, or of a cellulose derivative, for example of carboxymethyl-cellulose (CMC), are initially introduced into a vessel which can preferably be screwed shut and stirred to give the material according to the invention according to the embodiments described above after addition of the dispersant, in particular of patient's blood, optionally while carrying out an incubation.

For the further stabilization of the material or molded article according to the invention, it may be preferred additionally to add a preferably chemical crosslinker, in particular a carbodiimide, preferably N-(dimethylaminopropyl)-N′-ethylcarbodimide (EDC), or glutaraldehyde, the crosslinking being possible before or after the possible addition of a preferably structure-imparting filler.

For the provision of a molded article, the removal of the dispersant is preferably carried out by freeze-drying, preferably in a shape-imparting environment. Advantageously, the shape-imparting environment is the shaped hollows of a well plate, in particular of a 24- or 96-well plate, in particular of polystyrene. By means of freeze-drying, it is possible, depending on the shapes of the wells, to produce different three-dimensional porous shaped articles, in particular bars, rings, cylinders, disks, cuboids or the like. By the choice of the rate and the temperature of the freeze-drying process, the pore size of the material according to the invention can be influenced in a particularly advantageous manner. Preferably, the suspension volumes transferred to the shaped hollows of a well plate are slowly frozen and freeze-dried in an oil pump vacuum at temperatures between −10° C. and −60° C., in particular at −50° C.

The invention moreover relates to a kit which consists essentially of a container which contains collagen containing an active substance complex and a gel-forming component. In another embodiment, the kit according to the invention consists of two containers, one container containing collagen containing an active substance complex and the other container containing a gel-forming component.

According to the invention, it can be provided for the collagen and/or the gel-forming component to be present in a preferably aqueous medium, in particular in a suspension or solution. Preferably, the kit contains an aqueous collagen suspension and an aqueous solution of the gel-forming component, which after homogeneous mixing afford the material according to the invention. For reasons of storability, it can be preferred for the collagen and/or the gel-forming component to be present in a solid, preferably pulverulent, form and optionally also as a mixture having a putty-like composition. In this way, the collagen and the gel-forming component can be stored for a relatively long period, for example 3 years, without adverse effects on their properties. The addition of blood, in particular patient's blood, or of a sterilized, preferably autoclaved, dispersant can preferably take place only immediately before the application of the material according to the invention.

Preferably, the kit is further characterized by a collagen and a gel-forming component, as are described in one of the preceding embodiments.

According to the invention, it can furthermore be provided for the container or the two containers of the kit according to the invention to be contained in a syringe cylinder of a single-chamber or dual-chamber syringe or to be formed by this. Preferably, the two-chamber syringe contains a device for the mixing of the collagen (containing active substance complex) and of the gel-forming component, in particular a mixing nozzle, which is preferably attachable to a dual-chamber syringe. Preferably, in the case of a dual-chamber syringe, one container contains an aqueous collagen suspension (containing active substance complex), in particular a COLLOSS® suspension or COLLOSS®E suspension, preferably COLLOSS®E suspension, and the other container contains an aqueous solution of the gel-forming component, in particular of a polysaccharide or polysaccharide derivative, in particular of a glycosaminoglycan (GAG), for example of hyaluronic acid or heparin, or of a cellulose derivative, for example of carboxymethylcellulose (CMC). By use of the dual-chamber syringe as intended, mixing of the suspension and of the solution occurs, and the resulting material according to the invention can be injected directly into the bone and/or cartilage defect to be treated.

The material according to the invention is outstandingly suitable, on account of its osteo-conductive and -inductive properties, which are distributed in a relatively large volume on account of a dispersant and of a gel-forming component contained in it, for the treatment of even large-area bone and/or cartilage defects. As a result of its adjustable shape stability, it is suitable in particular for the treatment of defects having poorly accessible cavities to be filled, in particular for the treatment of comminuted fractures. These advantages are supplemented by simple and safe handleability of the material according to the invention for the user, in particular the surgeon.

Further features and advantages of the invention follow from the subsequent description of preferred embodiments by means of examples in conjunction with the subclaims. Here, the individual features can in each case be realized per se or in combination with one another.

### EXAMPLE 1

20 mg of a lyophilizate of COLLOSS® (product of the Applicant, whose properties are explained in the description), are resuspended in 1 ml of sodium chloride (0.9%). A milky solution containing small flocks results, which are to be attributed to insoluble collagen aggregates. Subsequently, 1 ml of a 3% strength autoclaved CMC solution are added to this suspension and it is largely homogeneously mixed. In this way, a 1.5% CMC/COLLOSS® suspension (10 mg/ml of COLLOSS®) is formed in a 2 ml total volume. Optionally, different amounts of OSSAPLAST® (product of the Applicant, whose properties are explained in the description) can be added as a filler and likewise homogeneously mixed. Subsequently, the suspension is frozen at −50° C. for about 5 hours. Lyophilization overnight affords a mechanically strengthened, optically homogeneous molded article, which after addition of a liquid, for example water or physiological saline solution, is reconverted to a gel. This approach can also be carried out using a suspension of COLLOSS® instead of a lyophilizate as a starting material.

### EXAMPLE 2

200 mg of a CMC powder sterilized by gamma irradiation are initially introduced into a dish. Subsequently, 2 ml of a concentrated sterile COLLOSS® suspension having a DM value (dry matter value) of about 12 mg/g are added. The mixture obtained is stirred for several minutes with a sterile laboratory spatula until the formation of a homogeneous gel composition.

### EXAMPLE 3

2% w/v CMC powder is added to a concentrated COLLOSS® suspension having a DM value (dry matter value) of about 12 mg/g. Subsequently, the mixture is homogeneously mixed with a sterile spatula until the formation of a gel. After this, 5 ml of the gel are transferred to a syringe and lyophilized. The lyophilized COLLOSS®-CMC gels are subsequently compressed from 5 ml to about 1 ml (concentration factor: 5×). The syringe is cut open at the top using a scalpel and the press pellet thus obtained is transferred to a glass bottle. Subsequently, 1 ml of completely demineralized water is added by means of a syringe. During the gel formation, the pellet is stirred several times with a spatula at room temperature in the course of about 20 min. A very putty-like milk-colored and nontransparent gel results here.

### EXAMPLE 4

200 mg of sterile CMC powder and 100 mg of sterile collagen type 1 powder (manufacturer: PONETI) are initially introduced under sterile conditions into a vessel (vial) which can be screwed shut and 20 mg of sterile COLLOSS® lyophilizate are added from another vessel (vial). Subsequently, 2 ml of patient's blood are added. The mixture obtained is agitated for 2 to 3 minutes. Optionally, the mixture is incubated in the closed vessel for 30 minutes and subsequently agitated again. A putty-like gel which is colored by blood and nontransparent results.

### EXAMPLE 5

20 mg of a lyophilizate of COLLOSS®E (product of the Applicant, whose properties are explained in the description), are resuspended in 1 ml of sodium chloride (0.9%). A milky solution containing small flocks results, which are to be attributed to insoluble collagen aggregates. Subsequently, 1 ml of a 3% strength autoclaved CMC solution are added to this suspension and it is largely homogeneously mixed. In this way, a 1.5% CMC/COLLOSS®E suspension (10 mg/ml of COLLOSS®E) is formed in a 2 ml total volume. Optionally, different amounts of OSSAPLAST® (product of the Applicant, whose properties are explained in the description) can be added as a filler and likewise homogeneously mixed. Subsequently, the suspension is frozen at −50° C. for about 5 hours. Lyophilization overnight affords a mechanically strengthened, optically homogeneous molded article, which after addition of a liquid, for example water or physiological saline solution, is reconverted to a gel. This approach can also be carried out using a suspension of COLLOSS®E instead of a lyophilizate as a starting material.

### EXAMPLE 6

200 mg of a CMC powder sterilized by gamma irradiation are initially introduced into a dish, Subsequently, 2 ml of a concentrated sterile COLLOSS®E suspension having a DM value (dry matter value) of about 12 mg/g are added. The mixture obtained is stirred for several minutes with a sterile laboratory spatula until the formation of a homogeneous gel composition.

### EXAMPLE 7

2% w/v CMC powder is added to a concentrated COLLOSS®E suspension having a DM value (dry matter value) of about 12 mg/g. Subsequently, the mixture is homogeneously mixed with a sterile spatula until the formation of a gel. After this, 5 ml of the gel are transferred to a syringe and lyophilized. The lyophilized COLLOSS®E-CMC gels are subsequently compressed from 5 ml to about 1 ml (concentration factor: 5×). The syringe is cut open at the top using a scalpel and the press pellet thus obtained is transferred to a glass bottle. Subsequently, 1 ml of completely demineralized water is added by means of a syringe. During the gel formation, the pellet is stirred several times with a spatula at room temperature in the course of about 20 min. A very putty-like milk-colored and nontransparent gel results here.

### EXAMPLE 8

200 mg of sterile CMC powder and 100 mg of sterile collagen type 1 powder (manufacturer: PONETI) are initially introduced under sterile conditions into a vessel (vial) which can be screwed shut and 20 mg of sterile COLLOSS®E lyophilizate are added from another vessel (vial). Subsequently, 2 ml of patient's blood are added. The mixture obtained is agitated for 2 to 3 minutes. Optionally, the mixture is incubated in the closed vessel for 30 minutes and subsequently agitated again. A putty-like gel which is colored by blood and nontransparent results.

1. A gelatinous material for filling bone and/or cartilage defects, comprising at least one gel-forming component and collagen in at least one liquid dispersant, characterized in that the collagen contains an active substance complex.

2. The material as claimed in claim 1, characterized in that the dispersant in water or an aqueous medium, in particular a physiological saline solution or blood.

3. The material as claimed in claim 1, characterized in that the gel-forming component is a hydrophilic polymer.

4. The material as claimed in claim 3, characterized in that the polymer is a polysaccharide or polysaccharide derivative, in particular a glycosaminoglycan (GAG), preferably hyaluronic acid or heparin, or a cellulose derivative, preferably carboxymethylcellulose (CMC), hydroxyethylcellulose (HEC), hydroxypropylcellulose (HPC) or ethylhydroxyethylcellulose (EHEC).

5. The material as claimed in claim 1, characterized in that the concentration of the gel-forming component, in particular of the polysaccharide or polysaccharide derivative, preferably of hyaluronic acid or carboxymethylcellulose (CMC), is between 0.5 and 25% by weight, in particular between 1 and 15% by weight, preferably preferably between 1 and 10% by weight, based on the total volume of the gelatinous material.

6. The material as claimed in claim 1, characterized in that the collagen is present in its native conformation.

7. The material as claimed in claim 1, characterized in that the collagen is present in denatured form, in particular acidically or basically treated form.

8. The material as claimed in claim 1, characterized in that the active substance complex is of natural origin.

9. The material as claimed in claim 1, characterized in that the active substance complex has osteoinductive properties.

10. The material as claimed in claim 8, characterized in that the active substance complex contains at least one recruitment and/or adhesion and/or growth and/or maturation factor.

11. The material as claimed in claim 1, characterized in that the active substance complex comprises a cytostatic and/or an antimicrobiotic, in particular antibiotic, active substance.

12. The material as claimed in claim 1, characterized in that it additionally contains at least one preferably structure-imparting filler.

13. The material as claimed in claim 12, characterized in that the filler is present as granules.

14. The material as claimed in claim 12, characterized in that the filler is bioresorbable.

15. The material as claimed in claim 12, characterized in that the filler is porous.

16. The material as claimed in claim 12, characterized in that the filler is tricalcium phosphate, hydroxyapatite (HA) or a light metal.

17. The material as claimed in claim 1, characterized in that it is preferably chemically crosslinked, in particular using a carbodiimide, in particular using N-(dimethyl-aminopropyl)-N′-ethylcarbodiimide (EDC), or using glutaraldehyde.

18. The material as claimed in claim 1, characterized in that it is sterilized, preferably by gamma irradiation and/or ethylene oxide fumigation, and/or aseptically treated and/or prepared.

19. The material as claimed in claim 1, characterized in that it is present in packaged form, in particular a sterile-packaged form.

20. A molded article for filling bone and/or cartilage defects, characterized in that it consists at least partially, preferably completely, of the material as claimed in claim 1, the dispersant being removed at least partially, preferably completely.

21. The molded article as claimed in claim 20, characterized in that it is freeze-dried for removal of the dispersant.

22. A process for the production of the material or molded article as claimed in claim 1, comprising the steps:
preparation of a mixture of a collagen containing an active substance complex, a gel-forming component and at least one dispersant,
optionally addition of a preferably structure-imparting filler, for the provision of the gelatinous material and optionally for the provision of the molded article,
removal of the dispersant from the gelatinous material, preferably in a shape-imparting environment.

23. The process as claimed in claim 22, characterized in that the mixture is prepared from a suspension of the collagen in the dispersant and/or a solution of the gel-forming component in the dispersant.

24. The process as claimed in claim 23, characterized in that the suspension is prepared from freeze-dried collagen.

25. The process as claimed in claim 23, characterized in that the suspension is prepared from extracted collagen.

26. The process as claimed in claim 22, characterized in that the collagen and/or the gel-forming component are present or prepared in solid form, preferably as a lyophilizate and/or powder.

27. The process as claimed in claim 22, characterized in that the removal of the dispersant is carried out by freeze-drying.

28. A kit, consisting of a container, characterized in that the container contains collagen containing an active substance complex and a gel-forming component.

29. A kit, consisting essentially of two containers, characterized in that one container contains collagen containing an active substance complex and the other container contains a gel-forming component.

30. A kit as claimed in claim 28, characterized in that the collagen and/or the gel-forming component are present in a preferably aqueous medium, in particular in a suspension or solution.

31. The kit as claimed in claim 28, further characterized in that the collagen and a gel-forming component, are in at least one fluid dispersant.

32. The kit as claimed in claim 28, characterized in that the container or the two containers are contained in a syringe cylinder of a single-chamber or dual-chamber syringe or are formed by this.

33. The kit as claimed in claim 32, characterized in that the two-chamber syringe contains a device for the mixing of the collagen and of the gel-forming component, in particular a mixing nozzle, which is preferably attachable to the dual-chamber syringe.
